(19) United States
(12) Patent Application Publication
(10) Pub. No.: US 2019/0276786 A1
(43) Pub. Date: Sep. 12, 2019

(54) Multilayer Polymertic Containers for Bioreactors

(71) Applicant: FloDesign Sonics, Inc., Wilbraham, MA (US)

(72) Inventors: Bart Lipkens, Bloomfield, CT (US); Rui Tostoes, Northampton, MA (US); Benjamin Ross-Johnsrud, Northampton, MA (US); Thomas J. Kennedy, III, Wilbraham, MA (US)

(21) Appl. No.: 16/298,973

(22) Filed: Mar. 11, 2019

Related U.S. Application Data
(60) Provisional application No. 62/741,956, filed on Oct. 5, 2018.
(60) Provisional application No. 62/641,237, filed on Mar. 9, 2018.

(51) Int. Cl. C12M 1/00; C08L 23/02; C08L 29/04

(52) U.S. Cl. CPC C12M 23/14; C08L 23/02; C08L 29/04; C12M 23/28; B01B 1/06; C08L 2314/02; C08L 2314/06; C08L 2201/14; C12M 47/10

(57) ABSTRACT

A bioreactor utilizing a multilayer disposable bag that may include at least one ultrasonic transducer that can acoustically generate a multi-dimensional standing wave. The standing wave can be used to retain cells in the bioreactor, and can also be utilized to dewater or further harvest product from the waste materials produced in a bioreactor.

### CROSS-REFERENCE TO RELATED APPLICATIONS

This application claims the benefit of U.S. Provisional Application Ser. No. 62/741,956, filed Oct. 5, 2018 and U.S. Provisional Application Ser. No. 62/641,237, filed Mar. 9, 2018, the entire contents of which are hereby incorporated herein by reference.

### BACKGROUND

Growth in the field of biotechnology has accelerated in the last decade with new processes, therapeutics and applications being brought to bear for the pharmacological industry. Great improvements in clinical trials, the processing parameters in bioreactors and filtration Improvements in equipment have allowed for larger volumes and lower cost for the production of biologically derived materials such as monoclonal antibodies and recombinant proteins. New materials have been incorporated into the bioprocesses to improve throughput and reduce costs.

The initial bioreactors that were utilized in the bioprocessing industry were stainless steel. These bioreactors worked very well but operated with downtime due to the clean in place (CIP) process involved in the continuous process of producing biomolecules. The CIP process also involves the use of manpower, steam, and cleaning agents to ensure the sterilization of the equipment.

### BRIEF DESCRIPTION

The present disclosure relates, in various embodiments, to multilayer polymeric, flexible containers which includes a plurality of layers and may have an interior volume ranging from 0.01-1000 liters. These polymeric, flexible containers are available in a variety of sizes to accommodate different uses. A biocompatible product-contacting layer of the interior of flexible container may be formed of a low-density polyethylene, very low-density polyethylene, ethylene vinyl acetate copolymer, polyester, polyamide, polyvinylchloride, polypropylene, polyfluoroethylene, polyvinylidenefluoride, polyurethane or fluoroethylenepropylene, and combinations thereof as examples. A gas and water vapor barrier layer may also be formed of an ethylene/vinyl alcohol copolymer mixture within a polyamide or an ethylene vinyl acetate copolymer. The polymeric flexible container may include a layer with high mechanical strength (e.g. a polyamide or polyester), and an external layer with insulating effect to heat welding, for example, polyester. The layers may be compatible with warm and cold conditions and may be able to withstand ionizing irradiation, such as gamma radiation, for sterilization purposes. The polymeric flexible container may also have a large surface area to volume ratio, and a relatively thin wall thus promoting heat transfer therethrough when received in temperature control unit. One example of materials useful for formulation of flexible container is described in U.S. Pat. No. 5,988,422 to Vallot, the entire subject matter of which is hereby incorporated herein by reference. The polymeric flexible container may be disposable, thus promoting ease of use and preventing cross-contamination of the interior of the polymeric flexible container which might result when reusing other types of containers.

A multi-constituent catalyst system they be utilized to produce the polyolefin materials. Such a catalyst system is comprised of a Ziegler-Natta catalyst composition including a magnesium and titanium containing procatalyst and a cocatalyst. The procatalyst is a Ziegler Natta catalyst including a titanium compound supported on MgCl₂. The cocatalyst is a triethylaluminum (TEA). The procatalyst may have a Ti:Mg ratio between 1.0:40 to 5.0:40, for example, 3.0:40. The procatalyst and the cocatalyst components can be contacted either before entering the reactor or in the reactor. The procatalyst may, for example, be any other titanium based Ziegler Natta catalyst. The Al:Ti molar ratio of cocatalyst component to procatalyst component can be from 0.5:1 to 10:1, for example 3:1.

The multi-constituent catalyst system includes a Ziegler-Natta catalyst composition including a magnesium and titanium containing procatalyst and a cocatalyst. The procatalyst may, for example, comprise the reaction product of magnesium dichloride, an alkylaluminum dihalide, and a titanium alkoxide. The procatalyst may comprise the reaction product of:
(A) a magnesium halide prepared by contacting: (1) at least one hydrocarbon soluble magnesium component represented by the general formula R″R′Mg.xAIR′3 wherein each R″ and R′ are alkyl groups; (2) at least one non-metallic or metallic halide source under conditions such that the reaction temperature does not exceed a temperature in the range of from 20 to 40, for example, it does not exceed about 40.degree. C.; or in the alternative, it does not exceed about 35.degree. C.;
(B) at least one transition metal compound represented by the formula Tm(OR)y Xy-x wherein Tm is a metal of Groups IVB, VB, VIB, VIIB or VIII of the Periodic Table; R is a hydrocarbyl group having from 1 to about 20, for example from 1 to about 10 carbon atoms; X is a halide, and y and x are integers and their sum is equal to 4, and
(C) an additional halide source to provide the desired excess X:Mg ratio; wherein additional halide source may be an organo halide compound of Group IIIA metal including, for example, those represented by the formula R′_{y}MX_{z}; wherein M is a metal from Group IIIA of the Periodic Table of Elements, for example aluminum or boron; each R′ is independently an alkyl group having from 1 to 20, for example from 1 to 10, or in the alternative, from 2 to 8, carbon atoms; X is a halogen atom, for example chlorine; y and z each independently have a value from 1 to a value equal to the valence of M. Particularly suitable organo halide compounds include, for example, ethylaluminum dichloride, ethylaluminum sequichloride; diethylaluminum chloride; isobutylaluminum dichloride; diisobutylaluminum chloride; octylaluminum dichloride; and combinations of 2 or more thereof.

Particularly suitable transition metal compounds include, for example, titanium tetrachloride, titanium trichloride, tetra(isopropoxy)-titanium, tetrabutoxytitanium, diethoxytitanium dibromide, dibutoxytitanium dichloride, tetraphenoxytitanium, tri-isopropoxy vanadium oxide, zirconium tetra-n-propoxide, mixtures thereof and the like.

Another catalyst system that is utilized to produce polyolefins is known as a coordination catalyst or a metallocene catalyst. The metallocene catalyst system typically produces a very linear polyolefin. It also produces a polyolefin with rather low density, down to 0.85 g per cc. These catalyst offers single site polymerization for polyolefins. The precision of these catalyst give the polymers high strength and clarity. Metallocene catalyst are metal complexes with two cyclopentadienyl (Cp) or substituted Cp groups. Standard Ziegler Natta catalyst, in contrast, are typically built from titanium and chlorine.

The general name metallocene is derived from ferrocene, the first metallocene type catalyst. Metallocene catalyst contains a transition metal and 2 cyclopentadienyl ligands coordinated in a sandwich structure, i.e., The two cyclopentadienyl anions are on parallel planes with equal bond lengths and strengths. Other types of metallocene catalyst utilizes zirconium as the transition metal.

Disclosed in various embodiments is a system comprising a bioreactor and a filtering device. The bioreactor may includes a reaction volume, an agitator, a feed inlet, and an outlet. The agitator may be comprised of an ultrasonic device that utilizes acoustic streaming for mixing. The filtering device comprises: an inlet fluidly connected to the bioreactor outlet for receiving fluid from the bioreactor; a flow chamber through which the fluid can flow; and at least one ultrasonic transducer and a reflector located opposite the at least one ultrasonic transducer, the at least one ultrasonic transducer being driven to produce a multi-dimensional standing wave in the flow chamber. The filtering device may or may not be coupled to the bioreactor.

The container may be comprised of a multilayer construction of films where a random layer construction of metallocene catalyzed Polyolefins, a Ziegler Natta catalyzed polyolefin, a non-hydrolyzed polyvinyl acetate, a hydrolyzed polyvinyl acetate, and/or a polyester where at least one polymer layer is treated with a plasma or corona treatment.

The layered polymeric construction they also have a total acoustic impedance of less than 3.0 Pa seconds per cubic meter (Pa·s/m3).

The layered polymeric construction may also contain at least one layer that is foamed or cellular.

The filtering or trapping device for the bioreactor may also consist of a flow chamber with one or more ultrasonic transducers and reflectors incorporated into the flow chamber. The reflectors are set up opposite the ultrasonic transducers and the ultrasonic transducers are electronically driven to form a multi-dimensional acoustic standing wave in the flow chamber. The multilayer bioreactor bag may be attached to the filtering or trapping device. The flow chamber may also be attached to the bioreactor bag. The filtering or trapping device may also be located internal to the bioreactor bag.

The flow chamber may be made from a rigid material, such as a plastic, glass or metal container. The flow chamber may alternatively be in the form of a flexible polymeric bag or pouch that is capable of being sealed and removed from the recirculation path between the bioreactor outlet through the external filtering device and a recycle inlet of the bioreactor. This flexible polymeric bag or pouch may be located between an ultrasonic transducer and a reflector such that a multi-dimensional acoustic standing wave may be generated interior to the flexible polymeric bag or pouch.

The filtering device may further comprise a product outlet through which desired product, such as expanded cells, viruses, exosomes, or phytochemicals are recovered. The filtering device can also further comprise a recycle outlet for sending fluid back to the bioreactor.

The multi-dimensional standing wave may have an axial force component and a lateral force component which are of the same order of magnitude. The bioreactor can be operated as a perfusion bioreactor.

In particular embodiments, the ultrasonic transducer comprises a piezoelectric material that can vibrate in a higher order mode shape. The piezoelectric material may have a rectangular shape.

The ultrasonic transducer may comprise: a housing having a top end, a bottom end, and an interior volume; and a crystal at the bottom end of the housing having an exposed exterior surface and an interior surface, the crystal being able to vibrate when driven by a voltage signal. In some embodiments, a backing layer contacts the interior surface of the crystal, the backing layer being made of a substantially acoustically transparent material. The substantially acoustically transparent material can be balsa wood, cork, or foam. The substantially acoustically transparent material may have a thickness of up to 1 inch. The substantially acoustically transparent material can be in the form of a lattice. In other embodiments, an exterior surface of the crystal is covered by a wear surface material with a thickness of a half wavelength or less, the wear surface material being a urethane, epoxy, or silicone coating. In yet other embodiments, the crystal has no backing layer or wear layer.

The ultrasonic transducer may also comprise a piezoelectric material that is polymeric such as polyvinylidene fluoride (PVDF). The PVDF may be excited at higher frequencies up to the hundreds of megahertz range such that very small particles may be trapped by the acoustic standing wave.

The multi-dimensional standing wave can be a three-dimensional standing wave.

The reflector may have a non-planar surface.

The product outlet of the filtering device may lead to a further process such as cell washing, cell concentration or cell fractionation. Such processes may be applied when the recovered product is biological cells such as T cells, B cells and NK cells. In certain embodiments, the cells used to produce viruses or exosomes are Chinese hamster ovary (CHO) cells, NSO hybridoma cells, baby hamster kidney (BHK) cells, or human cells. The use of mammalian cell cultures including the aforementioned cell types has proven to be a very efficacious way of producing/expressing the recombinant proteins and monoclonal antibodies used in today's pharmaceuticals. In some embodiments, the cells are plant cells that produce secondary metabolites and recombinant proteins and other phytochemicals.

These and other non-limiting characteristics are more particularly described below.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 illustrates a multilayer polymeric film.

FIG. 2 is an illustration of a multilayer polymeric extrusion head for co-extruding multilayer polymeric films.

FIG. 3 is a cross-sectional view that shows a multilayer extrusion head for a blown film process where a multilayer film is produced utilizing extrusion and blown air.

FIG. 4 shows a multilayer film with a mixture of polyolefins and other polymeric materials.

FIG. 5 shows a multilayer bioreactor bag situated in the bioreactor infrastructure for holding the bag in controlling the conditions within the bag.

FIG. 6 is a depiction of a multilayer polymeric bag reactor being utilized in a rocking bioreactor configuration.

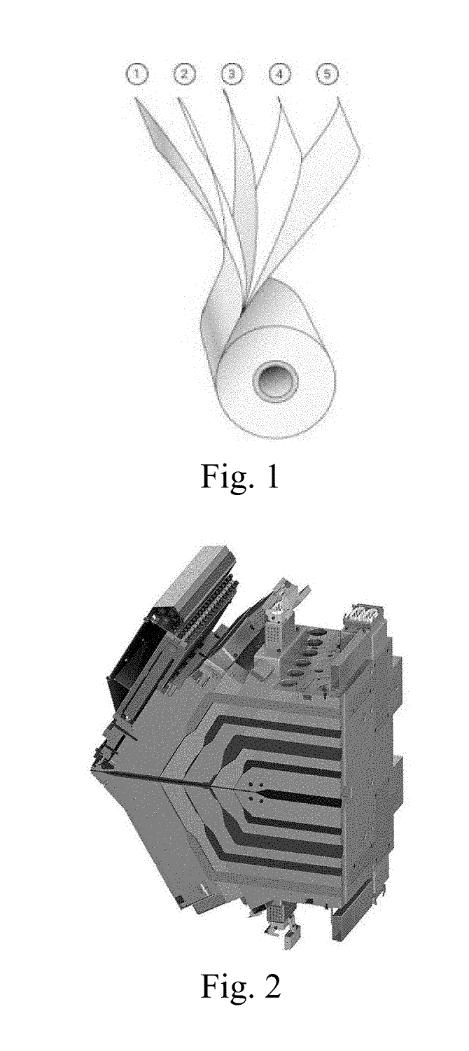

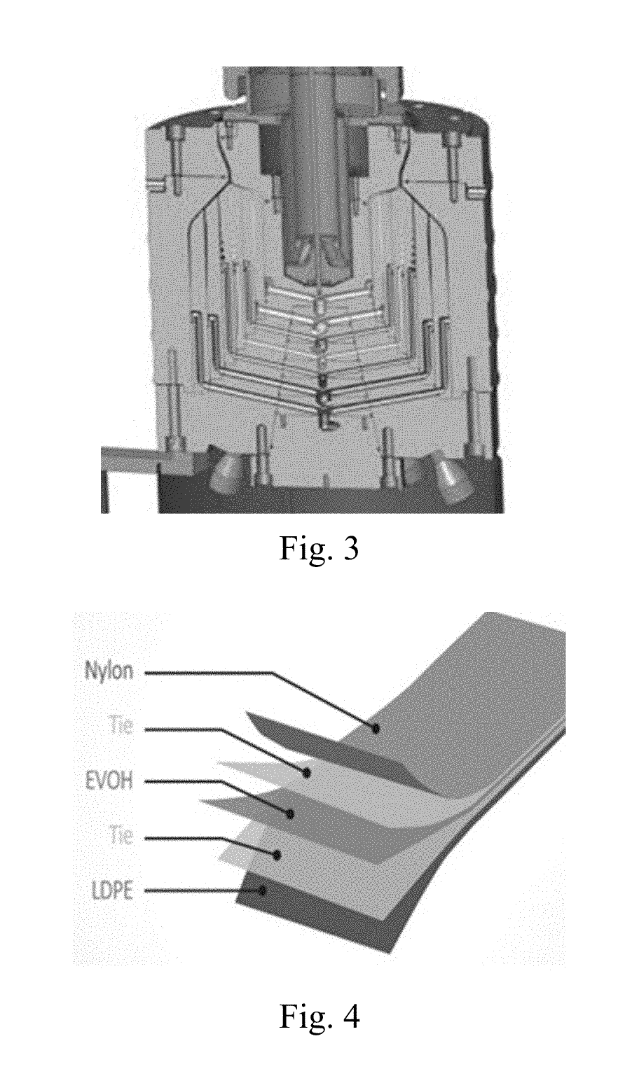

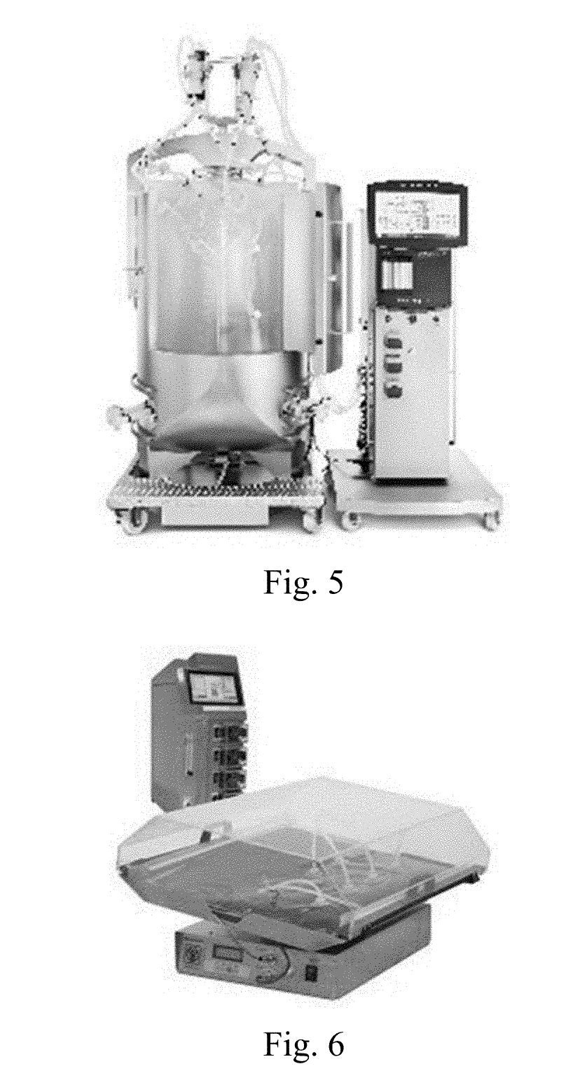

### DETAILED DESCRIPTION

The present disclosure may be understood more readily by reference to the following detailed description of desired embodiments and the examples included therein. In the following specification and the claims which follow, reference will be made to a number of terms which shall be defined to have the following meanings.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

The term “comprising” is used herein as requiring the presence of the named component and allowing the presence of other components. The term “comprising” should be construed to include the term “consisting of”, which allows the presence of only the named component, along with any impurities that might result from the manufacture of the named component.

Numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values). The endpoints of the ranges and any values disclosed herein are not limited to the precise range or value; they are sufficiently imprecise to include values approximating these ranges and/or values.

The modifier “about” used in connection with a quantity is inclusive of the stated value and has the meaning dictated by the context (for example, it includes at least the degree of error associated with the measurement of the particular quantity). When used in the context of a range, the modifier “about” should also be considered as disclosing the range defined by the absolute values of the two endpoints. For example, the range of “from about 2 to about 10” also discloses the range “from 2 to 10.”

It should be noted that many of the terms used herein are relative terms. For example, the terms “upper” and “lower” are relative to each other in location, i.e. an upper component is located at a higher elevation than a lower component in a given orientation, but these terms can change if the device is flipped. The terms “inlet” and “outlet” are relative to a fluid flowing through them with respect to a given structure, e.g. a fluid flows through the inlet into the structure and flows through the outlet out of the structure. The terms “upstream” and “downstream” are relative to the direction in which a fluid flows through various components, i.e. the flow fluids through an upstream component prior to flowing through the downstream component. It should be noted that in a loop, a first component can be described as being both upstream of and downstream of a second component.

The present application refers to “the same order of magnitude.” Two numbers are of the same order of magnitude if the quotient of the larger number divided by the smaller number is a value of at least 1 and less than 10.

The term “virus” refers to an infectious agent that can only replicate inside another living cell, and otherwise exists in the form of a virion formed from a capsid that surrounds and contains DNA or RNA, and in some cases a lipid envelope surrounding the capsid.

The term “exosome” refers to a vesicle, which has a lipid bilayer surrounding a core of fluid that contains proteins, DNA, and/or RNA.

The term “crystal” refers to a single crystal or polycrystalline material that is used as a piezoelectric material.

A new technology that is being utilized for bioreactors is the use of disposable plastic bags as the interior volume for these bioreactors. These plastic disposable bags utilize multilayer construction to provide a friendly environment for the biomaterials, structural strength materials to provide for a robust container in an industrial environment, gas permeability and low-cost materials to allow for reduced processing costs.

Discussed herein are implementations and techniques for disposable bioreactors. The disposable bioreactors may be implemented as disposable bioreactor bags. The disposable bioreactor bags can provide improved oxygen transmission, a friendlier cell environment, improved durability, improved compatibility with existing bioreactor systems and reduced cost.

The disposable bioreactors discussed herein may be combined with an ultrasonic device that can contribute to particle/cell manipulation. In some examples, the ultrasonic device can provide acoustophoresis action in a disposable bioreactor. Implementing an ultrasonic device in a disposable bioreactor permits a number of cell manipulation operations, including cell culturing or expansion, cell aggregation and cell separation internal to the disposable bioreactor, for example. Acoustophoresis is a low shear, low stress process that can be applied to cell manipulation, as opposed to the high shear, high cell stress environment observed with cell manipulation using physical mixing and physical filters.

Bioreactors are useful for making biomolecules such as recombinant proteins or monoclonal antibodies. Very generally, cells are cultured in a bioreactor vessel with media in order to produce the desired product, and the desired product is then harvested by separation from the cells and media. The use of mammalian cell cultures including Chinese hamster ovary (CHO), NSO hybridoma cells, baby hamster kidney (BHK) cells, and human cells has proven to be a very efficacious way of producing/expressing the recombinant proteins and monoclonal antibodies used in pharmaceuticals. Two general types of bioreactor processes exist: fed-batch and perfusion.

Bioreactors are also useful for making cell cultures such as T cells, B cells, NK cells and other components of the human immune system.

While fed-batch reactors are the norm currently, due mainly to the familiarity of the process to many scientists and technicians, perfusion technology is growing at a very fast clip. Many factors favor the use of a perfusion bioreactor process. The capital and start-up costs for perfusion bioreactors are lower, they use less resources and smaller capacity systems upstream and downstream, and the process uses smaller volumes and fewer seed steps than fed-batch methods. A perfusion bioreactor process also lends itself better to development, scale-up, optimization, parameter sensitivity studies, and validation.

Recent developments in bioreactors includes the development of disposable, multilayer polymeric bioreactor bags. These multilayer polymeric disposable bioreactor bags allow for a much quicker turnaround of the bioreactor materials in that the cleaning and preparation of the bioreactor is not necessary. Bioreactor bags may be utilized in fed-batch, batch and perfusion bioreactors.

A separate aspect of the use of high cell concentration bioreactors is the “dewatering” of the materials at the end of a bioreactor run. The “dewatering” or removal of interstitial fluid from a bioreactor sludge is important for improving the efficiency of recovery of the intended bioreactor product. Currently, high energy centrifuges with internal structures (known as disk stack centrifuges) are utilized to remove the interstitial fluid from the bioreactor sludge at the end of a run. The capital cost and operating costs for a disk stack centrifuge is high. A simpler method of removing the interstitial fluid from the remaining bioreactor sludge that can be performed without the high capital and operating costs associated with disk stack centrifuges is desirable. In addition, current methods of filtration or centrifugation can damage cells, releasing protein debris and enzymes into the purification process and increasing the load on downstream portions of the purification system.

The present disclosure relates to the use of multiple types of polyolefins and other polymers utilized in multiple layer bags for bioreactors. These are known in the industry as disposable bioreactors as opposed to the stainless steel or other components that are utilized to manufacture bioreactors in the current and prior use. The disposable bioreactors have an advantage over the former stainless-steel bioreactors in that the processes of cleaning place and sterilization are not needed due to the fact that the disposable bioreactors are single use and are disposed of after the culture or process they are involved in is completed.

Multiple types of materials are utilized in disposable bioreactor bags for various reasons. The inner layer of the disposable bioreactor bag should be innocuous to the development and expression of bio molecules from the cells held in the bioreactor bag. Chemical moieties from the internal surface of the bag can interfere with the biological processes of cell culturing and protein expression. Typically, polyolefins are utilized as the inner layer to prevent any aberrant biological processes or the restriction of biological processes by materials that may be on the surface of the inner part of the bioreactor bag. This would include monomers, catalysts, residual solvents, and the like.

Ensuring that the proper amount of oxygen is brought to the cells in the bioreactor bag, a layer of the bag may be utilized as an oxygen barrier and or transmission layer. This material may be a hydrolyzed polyvinyl acetate, also known as a polyvinyl alcohol. This polymer may be blended with other polymers to form a copolymer or terpolymer.

The durability and strength of the bioreactor bags is an important aspect of the bag as it is utilized through the manufacturing and disposal process. Polymeric layers which would give the bag strength and durability include polyamides, such as nylons, and polyesters, such as polyethylene terephthalate. These materials are typically the outer layers of the bioreactor bag.

Acoustophoresis is the manipulation of materials with acoustics. In some implementations, acoustophoresis provides for separation of materials in a low-power, no-pressure-drop, no-clog, solid-state approach to particle removal from fluid dispersions. For example, acoustophoresis can be used to achieve separations that are more typically performed with porous filters, but has fewer disadvantages associated with physical filters. In particular, the present disclosure provides filtering devices that are suitable for use with bioreactors and operate at the macro-scale for separations in flowing systems with high flow rates. The acoustophoretic filtering device is designed to create a high intensity multi-dimensional (e.g., three dimensional) ultrasonic standing wave that results in an acoustic radiation force that is larger than and can overcome the combined effects of fluid drag and buoyancy or gravity at certain flow rates, and is therefore able to trap (i.e., hold stationary) the suspended phase (i.e. cells) to allow more time for the acoustic wave to increase particle concentration, agglomeration and/or coalescence. Put another way, the radiation force of the acoustic standing wave(s) acts as a filter that prevents or retards targeted particles (e.g., biological cells) from crossing through the standing wave(s). The present systems have the ability to create ultrasonic standing wave fields that can trap particles in flow fields with a linear velocity ranging from 0.1 mm/sec to velocities exceeding 1 cm/s. As explained above, the trapping capability of a standing wave may be varied as desired, for example by varying the flow rate of the fluid, the acoustic radiation force, and the shape of the acoustic filtering device to maximize cell retention through trapping and settling. This technology offers a green and sustainable alternative for separation of secondary phases with a significant reduction in cost of energy. Excellent particle separation efficiencies have been demonstrated for particle sizes as small as one micron.

The ultrasonic standing waves can be used to trap, i.e., hold stationary, secondary phase particles (e.g. cells) in a host fluid stream (e.g. cell culture media). This is an important distinction from previous approaches where particle trajectories were merely altered by the effect of the acoustic radiation force. The scattering of the acoustic field off the particles results in a three dimensional acoustic radiation force, which acts as a three-dimensional trapping field. The acoustic radiation force is proportional to the particle volume (e.g. the cube of the radius) when the particle is small relative to the wavelength. It is proportional to frequency and the acoustic contrast factor. It also scales with acoustic energy (e.g. the square of the acoustic pressure amplitude). For harmonic excitation, the sinusoidal spatial variation of the force is what drives the particles to the stable positions within the standing waves. When the acoustic radiation force exerted on the particles is stronger than the combined effect of fluid drag force and buoyancy/gravitational force, the particle is trapped within the acoustic standing wave field. The action of the acoustic forces (i.e., the lateral and axial acoustic forces) on the trapped particles results in formation of tightly-packed clusters through concentration, clustering, clumping, agglomeration and/or coalescence of particles that, when reaching a critical size, settle continuously through enhanced gravity for particles heavier than the host fluid or rise out through enhanced buoyancy for particles lighter than the host fluid. Additionally, secondary inter-particle forces, such as Bjerkness forces, aid in particle agglomeration.

Generally, the 3-D standing wave(s) filtering system is operated at a voltage such that the protein-producing materials, such as Chinese hamster ovary cells (CHO cells), the most common host for the industrial production of recombinant protein therapeutics, are trapped in the ultrasonic standing wave, i.e., remain in a stationary position. Within each nodal plane, the CHO cells are trapped in the minima of the acoustic radiation potential. Most biological cell types present a higher density and lower compressibility than the medium in which they are suspended, so that the acoustic contrast factor between the cells and the medium has a positive value. As a result, the axial acoustic radiation force (ARF) drives the biological cells towards the standing wave pressure nodes. The axial component of the acoustic radiation force drives the cells, with a positive contrast factor, to the pressure nodal planes, whereas cells or other particles with a negative contrast factor are driven to the pressure anti-nodal planes. The radial or lateral component of the acoustic radiation force is the force that traps the cells. The radial or lateral component of the ARF is larger than the combined effect of fluid drag force and gravitational force. For small cells or emulsions the drag force F_{D} can be expressed as:

$${\vec{F}}_{D} = 4 {πμ}_{f} {R}_{p} ( {\vec{U}}_{f} - {\vec{U}}_{p} ) [ \frac{1 + \frac{3}{2} \hat{μ}}{1 + \hat{μ}} ] ,$$

where U_{f} and Uₚ are the fluid and cell velocity, Rₚ is the particle radius, μ_{f} and μₚ are the dynamic viscosity of the fluid and the cells, and \hat{μ}=μₚ/μ_{f} is the ratio of dynamic viscosities. The buoyancy force F_{B} is expressed as:

$${F}_{B} = \frac{4}{3} π {R}_{p}^{3} ( {ρ}_{f} - {ρ}_{p} ) .$$

To determine when a cell is trapped in the ultrasonic standing wave, the force balance on the cell may be assumed to be zero, and therefore an expression for lateral acoustic radiation force F_{LRF} can be found, which is given by:

F_{LRF}=F_{D}+F_{B}

For a cell of known size and material property, and for a given flow rate, this equation can be used to estimate the magnitude of the lateral acoustic radiation force.

One theoretical model that is used to calculate the acoustic radiation force is based on the formulation developed by Gor'kov. The primary acoustic radiation force F_{A} is defined as a function of a field potential U,

F_{A}=−∇(U),

where the field potential U is defined as

$$U = {V}_{0} [ \frac{〈 {p}^{2} 〉}{2 {ρ}_{f} {c}_{f}^{2}} {f}_{1} - \frac{3 {ρ}_{f} 〈 {u}^{2} 〉}{4} {f}_{2} ] ,$$

and f₁ and f₂ are the monopole and dipole contributions defined by

$${f}_{1} = 1 - \frac{1}{{Λσ}^{2}} , {f}_{2} = \frac{2 ( Λ - 1 )}{2 Λ + 1} ,$$

where p is the acoustic pressure, u is the fluid particle velocity, Λ is the ratio of cell density ρₚ to fluid density ρ_{f}, a is the ratio of cell sound speed cₚ to fluid sound speed c_{f}, Vₒ is the volume of the cell, and < > indicates time averaging over the period of the wave.

Gor'kov's theory is limited to particle sizes that are small with respect to the wavelength of the sound fields in the fluid and the particle, and it also does not take into account the effect of viscosity of the fluid and the particle on the radiation force. Additional theoretical and numerical models have been developed for the calculation of the acoustic radiation force for a particle without any restriction as to particle size relative to wavelength. These models also include the effect of fluid and particle viscosity, and therefore are a more accurate calculation of the acoustic radiation force. The models that were implemented are based on the theoretical work of Yurii Ilinskii and Evgenia Zabolotskaya as described in AIP Conference Proceedings, Vol. 1474-1, pp. 255-258 (2012). Additional in-house models have been developed to calculate acoustic trapping forces for cylindrical shaped objects, such as the “hockey pucks” of trapped particles in the standing wave, which closely resemble a cylinder.

The lateral force component of the total acoustic radiation force (ARF) generated by the ultrasonic transducer(s) of the present disclosure is significant and is sufficient to overcome the fluid drag force at linear velocities of up to 1 cm/s, and to create tightly packed clusters, and is of the same order of magnitude as the axial force component of the total acoustic radiation force. This lateral ARF can thus be used to retain cells in a bioreactor while the bioreactor process continues. This is especially true for a perfusion bioreactor.

The filtering devices of the present disclosure, which use ultrasonic transducers and acoustophoresis, can also improve the dewatering of the leftover material from a bioreactor batch (i.e bioreactor sludge), and thus reduce the use of or eliminate the use of disk stack centrifuges. This use or application of ultrasonic transducers and acoustophoresis simplifies processing and reduces costs.

In a perfusion bioreactor system, it is desirable to be able to filter and separate the cells and cell debris from the expressed materials that are in the fluid stream (i.e. cell culture media). The expressed materials are composed of biomolecules such as recombinant proteins or monoclonal antibodies, and are the desired product to be recovered.

An acoustophoretic filtering device can be used in at least two different ways. First, the standing waves can be used to trap the expressed biomolecules and separate this desired product from the cells, cell debris, and media. The expressed biomolecules can then be diverted and collected for further processing. Alternatively, the standing waves can be used to trap the cells and cell debris present in the cell culture media. The cells and cell debris, having a positive contrast factor, move to the nodes (as opposed to the anti-nodes) of the standing wave. As the cells and cell debris agglomerate at the nodes of the standing wave, there is also a physical scrubbing of the cell culture media that occurs whereby more cells are trapped as they come in contact with the cells that are already held within the standing wave. This generally separates the cells and cellular debris from the cell culture media. When the cells in the standing wave agglomerate to the extent where the mass is no longer able to be held by the acoustic wave, the aggregated cells and cellular debris that have been trapped can fall out of the fluid stream through gravity, and can be collected separately. To aid this gravitational settling of the cells and cell debris, the standing wave may be interrupted to allow all of the cells to fall out of the fluid stream that is being filtered. This process can be useful for dewatering. The expressed biomolecules may have been removed beforehand, or remain in the fluid stream (i.e. cell culture medium).

Desirably, the ultrasonic transducer(s) generate a multi-dimensional (e.g., three-dimensional) standing wave in the fluid that exerts a lateral force on the suspended particles to accompany the axial force so as to increase the particle trapping capabilities of the acoustophoretic filtering device. Typical results published in literature state that the lateral force is two orders of magnitude smaller than the axial force. In contrast, the technology disclosed in this application provides for a lateral force to be of the same order of magnitude as the axial force.

In the present disclosure, a perfusion bioreactor can also be used to generate cells that can subsequently be used for cell therapy. In this type of process, the biological cells to be used in the cell therapy are cultured in the bioreactor and expanded (i.e. to increase the number of cells in the bioreactor through cell reproduction). These cells may be lymphocytes such as T cells (e.g., regulatory T-cells (Tregs), Jurkat T-cells), B cells, or NK cells; their precursors, such as peripheral blood mononuclear cells (PBMCs); and the like. The cell culture media (aka host fluid), containing some cells, is then sent to a filtering device that produces an acoustic standing wave. A portion of the cells are trapped and held in the acoustic standing wave, while the remaining host fluid and other cells in the host fluid are returned to the bioreactor. As the quantity of trapped cells increases, they form larger clusters that will fall out of the acoustic standing wave at a critical size due to gravity forces. The clusters can fall into a product outlet outside a region of the acoustic standing wave, such as below the acoustic standing wave, from which the cells can be recovered for use in cell therapy. Only a small portion of the cells are trapped and removed from the bioreactor via the product outlet, and the remainder continue to reproduce in the bioreactor, allowing for continuous production and recovery of the desired cells.

In another application, acoustic standing waves are used to trap and hold biological cells and to separate viruses (e.g. lentiviruses) or exosomes that are produced by the biological cells. In these embodiments, the biological cells are returned to the bioreactor post-separation to continue production of viruses or exosomes.

In these applications, the acoustic filtering devices of the present disclosure can act as a cell retention device. The acoustic cell retention systems described herein operate over a range of cell recirculation rates, efficiently retain cells over a range of perfusion (or media removal) rates, and can be tuned to fully retain or selectively pass some percentage of cells through fluid flow rate, transducer power or frequency manipulation. Power and flow rates can all be monitored and used as feedback in an automated control system.

The cells of interest may also be held in the flow chamber of the external filtering device through the use of an acoustic standing wave such that other moieties may be introduced in close proximity to and for the purpose of changing the target cells. Such an operation would include the trapping of T cells and the subsequent introduction of modified lentivirus materials with a specific gene splice such that the lentivirus with a specific gene splice will transfect the T cell and generate a chimeric antigen receptor T cell also known as a CAR-T cell.

The acoustic filtering devices of the present disclosure are designed to maintain a high intensity three-dimensional acoustic standing wave. The device is driven by a function generator and amplifier (not shown). The device performance is monitored and controlled by a computer. It may be desirable, at times, due to acoustic streaming, to modulate the frequency or voltage amplitude of the standing wave. This modulation may be done by amplitude modulation and/or by frequency modulation. The duty cycle of the propagation of the standing wave may also be utilized to achieve certain results for trapping of materials. In other words, the acoustic beam may be turned on and shut off at different frequencies to achieve desired results.

The transducer design can affect performance of the system. A typical transducer is a layered structure with the ceramic crystal bonded to a backing layer and a wear plate. Because the transducer is loaded with the high mechanical impedance presented by the standing wave, the traditional design guidelines for wear plates, e.g., half wavelength thickness for standing wave applications or quarter wavelength thickness for radiation applications, and manufacturing methods may not be appropriate. Rather, in one embodiment of the present disclosure the transducers, there is no wear plate or backing, allowing the crystal to vibrate in one of its eigenmodes with a high Q-factor. The vibrating ceramic crystal/disk is directly exposed to the fluid flowing through the flow chamber.

Removing the backing (e.g. making the crystal air backed) also permits the ceramic crystal to vibrate at higher order modes of vibration with little damping (e.g. higher order modal displacement). In a transducer having a crystal with a backing, the crystal vibrates with a more uniform displacement, like a piston. Removing the backing allows the crystal to vibrate in a non-uniform displacement mode. The higher order the mode shape of the crystal, the more nodal lines the crystal has. The higher order modal displacement of the crystal creates more trapping lines, although the correlation of trapping line to node is not necessarily one to one, and driving the crystal at a higher frequency will not necessarily produce more trapping lines.

In some embodiments, the crystal may have a backing that minimally affects the Q-factor of the crystal (e.g. less than 5%). The backing may be made of a substantially acoustically transparent material such as balsa wood, foam, or cork which allows the crystal to vibrate in a higher order mode shape and maintains a high Q-factor while still providing some mechanical support for the crystal. The backing layer may be a solid, or may be a lattice having holes through the layer, such that the lattice follows the nodes of the vibrating crystal in a particular higher order vibration mode, providing support at node locations while allowing the rest of the crystal to vibrate freely. The goal of the lattice work or acoustically transparent material is to provide support without lowering the Q-factor of the crystal or interfering with the excitation of a particular mode shape. In some examples, the higher order mode shape describes a Bessel function.

Placing the crystal in direct contact with the fluid also contributes to the high Q-factor by avoiding the dampening and energy absorption effects of the epoxy layer and the wear plate. Other embodiments may have wear plates or a wear surface to prevent the PZT, which contains lead, contacting the host fluid. This may be desirable in, for example, biological applications such as separating blood. Such applications might use a wear layer such as chrome, electrolytic nickel, or electroless nickel. Chemical vapor deposition could also be used to apply a layer of poly(p-xylylene) (e.g. Parylene) or other polymer. Organic and biocompatible coatings such as silicone or polyurethane are also usable as a wear surface.

In some embodiments, the ultrasonic transducer has a 1 inch diameter and a nominal 2 MHz resonance frequency. Each transducer can consume about 28 W of power for droplet trapping at a flow rate of 3 GPM. This power usage translates to an energy cost of 0.25 kW hr/m³. This measure is an indication of the very low cost of energy of this technology. Desirably, each transducer is powered and controlled by its own amplifier. In other embodiments, the ultrasonic transducer uses a square crystal, for example with 1″×1″ dimensions. Alternatively, the ultrasonic transducer can use a rectangular crystal, for example with 1″×2.5″ dimensions. Power dissipation per transducer was 10 W per 1″×1″ transducer cross-sectional area and per inch of acoustic standing wave span in order to get sufficient acoustic trapping forces. For a 4″ span of an intermediate scale system, each 1″×1″ square transducer consumes 40 W. The larger 1″×2.5″ rectangular transducer uses 100 W in an intermediate scale system. The array of three 1″×1″ square transducers would consume a total of 120 W and the array of two 1″×2.5″ transducers would consume about 200 W. Arrays of closely spaced transducers represent alternate potential embodiments of the technology. Transducer size, shape, number, and location can be varied as desired to generate desired three-dimensional acoustic standing wave patterns.

The size, shape, and thickness of the transducer determine the transducer displacement at different frequencies of excitation, which in turn affects separation efficiency. Typically, the transducer is operated at frequencies near the thickness resonance frequency (half wavelength). Gradients in transducer displacement typically result in more trapping locations for the cells/biomolecules. Higher order modal displacements generate three-dimensional acoustic standing waves with strong gradients in the acoustic field in all directions, thereby creating equally strong acoustic radiation forces in all directions, leading to multiple trapping lines, where the number of trapping lines correlate with the particular mode shape of the transducer.

In the present system examples, the system is operated at a voltage such that the particles (i.e. biomolecules or cells) are trapped in the ultrasonic standing wave, i.e., remain in a stationary position. The particles are collected in along well defined trapping lines, separated by half a wavelength. Within each nodal plane, the particles are trapped in the minima of the acoustic radiation potential. The axial component of the acoustic radiation force drives particles with a positive contrast factor to the pressure nodal planes, whereas particles with a negative contrast factor are driven to the pressure anti-nodal planes. The radial or lateral component of the acoustic radiation force is the force that traps the particle. It therefore, for particle trapping, is larger than the combined effect of fluid drag force and gravitational force. In systems using typical transducers, the radial or lateral component of the acoustic radiation force is typically several orders of magnitude smaller than the axial component of the acoustic radiation force. However, the lateral force generated by the transducers of the present disclosure can be significant, on the same order of magnitude as the axial force component, and is sufficient to overcome the fluid drag force at linear velocities of up to 1 cm/s.

The lateral force can be increased by driving the transducer in higher order mode shapes, as opposed to a form of vibration where the crystal effectively moves as a piston having a uniform displacement. The acoustic pressure is proportional to the driving voltage of the transducer. The electrical power is proportional to the square of the voltage. The transducer is typically a thin piezoelectric plate, with electric field in the z-axis and primary displacement in the z-axis. The transducer is typically coupled on one side by air (i.e. the air gap within the transducer) and on the other side by the fluid of the cell culture media. The types of waves generated in the plate are known as composite waves. A subset of composite waves in the piezoelectric plate is similar to leaky symmetric (also referred to as compressional or extensional) Lamb waves. The piezoelectric nature of the plate typically results in the excitation of symmetric Lamb waves. The waves are leaky because they radiate into the water layer, which result in the generation of the acoustic standing waves in the water layer. Lamb waves exist in thin plates of infinite extent with stress free conditions on its surfaces. Because the transducers of this embodiment are finite in nature, the actual modal displacements are more complicated.

The transducers are driven so that the piezoelectric crystal vibrates in higher order modes of the general formula (m, n), where m and n are independently 1 or greater. Generally, the transducers will vibrate in higher order modes than (2,2). Higher order modes will produce more nodes and antinodes, result in three-dimensional standing waves in the fluid layer, characterized by strong gradients in the acoustic field in all directions, not only in the direction of the standing waves, but also in the lateral directions. As a consequence, the acoustic gradients result in stronger trapping forces in the lateral direction.

In embodiments, the pulsed voltage signal driving the transducer can have a sinusoidal, square, sawtooth, or triangle waveform; and have a frequency of 500 kHz to 10 MHz. The pulsed voltage signal can be driven with pulse width modulation, which produces any desired waveform. The pulsed voltage signal can also have amplitude or frequency modulation start/stop capability to eliminate streaming.

The transducer(s) is/are used to create a pressure field that generates forces of the same order of magnitude both orthogonal to the standing wave direction and in the standing wave direction. When the forces are roughly the same order of magnitude, particles of size 0.1 microns to 300 microns will be moved more effectively towards regions of agglomeration (“trapping lines”). Because of the equally large gradients in the orthogonal acoustophoretic force component, there are “hot spots” or particle collection regions that are not located in the regular locations in the standing wave direction between the transducer and the reflector. Hot spots are located in the maxima or minima of acoustic radiation potential. Such hot spots represent particle collection locations which allow for better wave transmission between the transducer and the reflector during collection and stronger inter-particle forces, leading to faster and better particle agglomeration.

TABLE 1
2.5″ × 4″ System results at 15 L/hr Flow rate

| Frequency (MHz) | 30 Watts | 37 Watts | 45 Watts |
|---|---|---|---|
| 2.2211 | 93.9 | 81.4 | 84.0 |
| 2.2283 | 85.5 | 78.7 | 85.4 |
| 2.2356 | 89.1 | 85.8 | 81.0 |
| 2.243 | 86.7 | — | 79.6 |

In biological applications, all of the parts of the system (e.g. the flow chamber, tubing leading to and from the bioreactor or filtering device, the sleeve containing the ultrasonic transducer and the reflector, the temperature-regulating jacket, etc.) can be separated from each other and be disposable. Thus use of acoustic separation can obtain improved separation of the CHO cells without lowering the viability of the cells, which is a significant advantage over centrifuges and physical or membrane filters. The transducers may also be driven to create rapid pressure changes to prevent or clear blockages due to agglomeration of CHO cells. The frequency of the transducers may also be varied to obtain optimal effectiveness for a given power.

The acoustophoretic separators/filtering devices of the present disclosure can be used in a filter “train,” in which multiple different filtration steps are used to clarify or purify an initial fluid/particle mixture to obtain the desired product and manage different materials from each filtration step. Each filtration step can be designed and implemented to remove a particular material, improving the overall efficiency of the clarification process. An individual acoustophoretic device can implement one or multiple filtration steps. For example, each individual ultrasonic transducer within a particular acoustophoretic device can be operated to trap materials within a given particle range. The acoustophoretic device can be operated to rapidly remove large quantities of particulate material, reducing the burden on subsequent downstream filtration steps/stages. Additional filtration steps/stages can be placed upstream or downstream of the acoustophoretic device, such as physical filters or other filtration mechanisms known in the art, such as depth filters (e.g., polymeric morphology, matrix media adsorption), sterile filters, crossflow filters (e.g., hollow fiber filter cartridges), tangential flow filtration cassettes, adsorption columns, separation columns (e.g., chromatography columns), or centrifuges. Multiple acoustophoretic devices can be used as well. Desirable biomolecules or cells can be recovered/separated after such filtration/purification, as explained herein.

A disposable bioreactor vessel can be combined with an ultrasonic transducer. The disposable bioreactor vessel can be a multilayer bioreactor bag. The ultrasonic transducer can be incorporated directly into the bioreactor bag.

The devices discussed herein that include ultrasonic transducers can be implemented to perform a number of different operations, including acoustophoretic separators, collectors, filters, particle/cell retention, and are referred to collectively herein as acoustic devices. The outlets of the acoustic devices of the present disclosure (e.g., product outlet, recycle outlet) can be fluidly connected to any other filtration step or filtration stage. The inlets of the acoustic devices of the present disclosure can be fluidly connected to any other filtration step or filtration stage. The additional filtration steps/stages can be located upstream (e.g., between the acoustophoretic separators(s) and the bioreactor), downstream, or both upstream and downstream of the acoustic devices. The acoustic devices of the present disclosure can be used in a system with as few or as many filtration stages/steps located upstream or downstream, in series, parallel or recirculation paths thereof as is desired. The present systems can include a bioreactor, an acoustic device, and multiple filtrations stages/steps located upstream and/or downstream of the acoustic device, with the filtrations stage(s) and acoustic devices being fluidly connected to one another.

For example, when it is desired that the system include a filtration stage (e.g., a porous filter) located upstream of the acoustic device, the outlet of the bioreactor can lead to an inlet of the porous filter and the outlet of the porous filter can lead to an inlet of the acoustic device, with the porous filter pre-processing the fluid therein. As another example, when it is desired that the system include a filtration stage (e.g., a separation column) located downstream of the acoustic device, the outlet of the bioreactor can lead to an inlet of the acoustic device and the outlet of the acoustic device can lead to an inlet of the separation column, with the separation column further processing the fluid therein.

Such filtration steps/stages can include various methods such as an additional acoustic device, or physical filtration means known in the art, such as depth filtration, sterile filtration, size exclusion filtration, or tangential filtration. Depth filtration uses physical porous filtration mediums that can retain material through the entire depth of the filter. In sterile filtration, membrane filters with extremely small pore sizes are used to remove microorganisms and viruses, generally without heat or irradiation or exposure to chemicals. Size exclusion filtration separates materials by size and/or molecular weight using physical filters with pores of given size. In tangential filtration, the majority of fluid flow is across the surface of the filter, rather than into the filter.

Chromatography can also be used, including cationic chromatography columns, anionic chromatography columns, affinity chromatography columns, mixed bed chromatography columns. Other hydrophilic/hydrophobic processes can also be used for filtration purposes.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Specific details are given in the description to provide a thorough understanding of example configurations (including implementations). However, configurations may be practiced without these specific details. For example, well-known processes, structures, and techniques have been shown without unnecessary detail to avoid obscuring the configurations. This description provides example configurations only, and does not limit the scope, applicability, or configurations of the claims. Rather, the preceding description of the configurations provides a description for implementing described techniques. Various changes may be made in the function and arrangement of elements without departing from the spirit or scope of the disclosure.

Also, configurations may be described as a process that is depicted as a flow diagram or block diagram. Although each may describe the operations as a sequential process, many of the operations can be performed in parallel or concurrently. In addition, the order of the operations may be rearranged. A process may have additional stages or functions not included in the figure.

Having described several example configurations, various modifications, alternative constructions, and equivalents may be used without departing from the spirit of the disclosure. For example, the above elements may be components of a larger system, wherein other structures or processes may take precedence over or otherwise modify the application of the invention. Also, a number of operations may be undertaken before, during, or after the above elements are considered. Accordingly, the above description does not bound the scope of the claims.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

The present disclosure has been described with reference to exemplary embodiments. Modifications and alterations will occur to others upon reading and understanding the preceding detailed description. It is intended that the present disclosure be construed as including all such modifications and alterations insofar as they come within the scope of the appended claims or the equivalents thereof.

1. A system comprising:
a bioreactor with a flexible polymeric bag with multiple layers of polyolefins comprising Ziegler Natta catalyzed polyolefins and metallocene catalyzed polyolefins;
an acoustic device downstream of and fluidly connected to the bioreactor, comprising:
a flow chamber; and
an ultrasonic transducer; and
a downstream filtration stage downstream of and fluidly connected to the acoustic device.

2. The system of claim 1, wherein the downstream filtration stage is selected from the group consisting of depth filters, sterile filters, and crossflow filters.

3. The system of claim 1, wherein the downstream filtration stage isolates the desired product by size exclusion filtration.

4. The system of claim 1, wherein the downstream filtration stage includes a plurality of filtration stages arranged in series.

5. The system of claim 4, wherein at least one of the plurality of filtration stages is a separation column and another of the plurality of filtration stages is a porous filter, the porous filter located upstream of the separation column and fluidly connected thereto.

6. The system of claim 1, wherein the multi-dimensional standing wave has an axial force component and a lateral force component which are of the same order of magnitude.

7. The system of claim 1, wherein the downstream filtration stage is a second acoustophoretic separator comprising an ultrasonic transducer-reflector pair driven to produce a multi-dimensional standing wave in the second acoustophoretic separator that is adapted to trap desired product that is not trapped in the first acoustophoretic separator.

8. The system of claim 1, wherein the bioreactor is a perfusion bioreactor.

9. The system of claim 1, wherein the multi-dimensional standing wave has an axial force component and a lateral force component which are of the same order of magnitude.

10. A process for growing a cell culture, comprising:
seeding a cell culture in a media in a disposable bioreactor;
applying the cell culture to a multi-dimensional acoustic wave to separate cells from the media.

11. The process of claim 10, wherein the disposable bioreactor further comprises a multilayer polymeric bioreactor bag.

12. A system comprising:
a multilayer polymeric bioreactor bag;
an upstream filtration stage fluidly connected to the bioreactor and upstream of an acoustophoretic separator; and
the acoustophoretic separator fluidly connected to and downstream of the upstream filtration stage, the acoustophoretic separator comprising:
a flow chamber; and
an ultrasonic transducer configured to launch a multi-dimensional acoustic wave in the flow chamber; and
a recycle outlet downstream of the flow chamber connected to a recycle inlet of the reaction vessel, for sending the associated fluid containing cells back to the bioreactor.

13. The system of claim 12, wherein the upstream filtration stage is selected from the group consisting of depth filters, sterile filters, and crossflow filters.

14. The system of claim 12, wherein the upstream filtration stage isolates the desired product by size exclusion filtration.

15. The system of claim 12, wherein the upstream filtration stage includes a plurality of filtration stages arranged in series.

16. The system of claim 15, wherein at least one of the plurality of filtration stages is a separation column and another of the plurality of filtration stages is a porous filter, the porous filter located upstream of the separation column and fluidly connected thereto.

17. The system of claim 12, wherein the multi-dimensional standing wave has an axial force component and a lateral force component which are of the same order of magnitude.

18. The system of claim 12, wherein the upstream filtration stage is a second acoustophoretic separator comprising an ultrasonic transducer-reflector pair driven to produce a multi-dimensional standing wave in the second acoustophoretic separator.

19. The system of claim 12, wherein the bioreactor is a perfusion bioreactor.

20. The system of claim 12, wherein the multi-dimensional standing wave has an axial force component and a lateral force component which are of the same order of magnitude.
